(19) United States
(12) Patent Application Publication
(10) Pub. No.: US 2015/0218553 A1
(43) Pub. Date: Aug. 6, 2015

(54) Screening Polynucleotide Libraries For Variants That Encode Functional Proteins

(71) Applicant: NOVOZYMES A/S, Bagsværd (DK)

(72) Inventors: Robert Blazej, San Francisco, CA (US); Nicholas Toriello, Woodside, CA (US); Charles Emrich, San Francisco, CA (US)

(73) Assignee: Novozymes A/S, Bagsværd (DK)

(21) Appl. No.: 14/429,117

(22) PCT Filed: Sep. 20, 2013
(86) PCT No.: PCT/US13/60954

§ 371 Date: Mar. 18, 2015

Related U.S. Application Data
(60) Provisional application No. 61/703,566, filed on Sep. 20, 2012.

(51) Int. Cl. C12N 15/10

(52) U.S. Cl. CPC C12N 15/1086

(57) ABSTRACT

The present invention provides methods based on screening expressed polynucleotide libraries for soluble proteins.

### CROSS-REFERENCE TO RELATED APPLICATIONS

This application claims the benefit of U.S. provisional application no. 61/703,566, filed Sep. 20, 2012, which is hereby incorporated by reference in its entirety.

### STATEMENT AS TO RIGHTS TO INVENTIONS MADE UNDER FEDERALLY SPONSORED RESEARCH AND DEVELOPMENT

Not applicable.

### FIELD OF THE INVENTION

The present invention relates to the fields of microbiology, molecular biology, and protein biochemistry. More particularly, it relates to compositions and methods for analyzing and enriching polynucleotide libraries for variants that encode for functional proteins.

### BACKGROUND OF THE INVENTION

The screening of polynucleotide libraries for variants that encode preferred protein activities is a central enterprise in biotechnology (See, e.g., Jäckel and Hilvert, Curr Opin Biotechnol 21, 753-759 (2010)). Unfortunately, as library diversity increases, there is an exponential decrease in the number of active variants in the library (See, e.g., Guo et al., PNAS (2004); Bloom et al., PNAS (2005)). This inverse relationship results in the inefficient and costly screening of polynucleotide libraries. Since only a small fraction of the library encodes for functional proteins, it is often necessary to screen thousands, millions, or even billions of variants in order to identify desired clones. Screening is often slow and costly because it typically requires custom host cell transformation, protein expression and frequently purification and quantitation, specific reaction with substrate or ligand, signal detection and quantitation.

Many methods and extensive robotic automation have been developed to facilitate library screening efforts (See, e.g., Maerkl, Curr Opin Biotechnol 22, 59-65 (2011); Goddard and Reymond, Curr Opin Biotechnol 15, 314-322 (2004); Wahler and Reymond, Curr Opin Biotechnol 12, 535-544 (2001)). Nonetheless, screening throughput is insufficient and remains costly because promising variants must be identified from a huge pool of possibilities. For example, mutating two random amino acids in a 100-amino acid protein results in a library containing 1.98×10⁶ unique members (See, e.g., Dietrich et al, Ann Rev Biochem 79, 563-590 (2010)). A central limitation of all screening approaches is that they must be customized both to the activity or interaction of the target protein with specific reactants and to the means of detecting and isolating positive variants.

### SUMMARY OF THE INVENTION

In various aspects, the invention(s) contemplated herein may include, but need not be limited to, any one or more of the following embodiments.

### Embodiment 1

A method of enriching a plurality of polynucleotides for polynucleotides likely to encode functional polypeptides and screening the enriched plurality, the method including: (a) providing a plurality of polynucleotides encoding variants of a polypeptide, wherein at least some of the polynucleotides encode a polypeptide having at least one activity; (b) producing polypeptides from the polynucleotides; (c) determining whether the polypeptides are soluble; (d) selecting the polynucleotides that encode soluble polypeptides to form an enriched plurality of polynucleotides; and (e) screening the enriched plurality for polynucleotides encoding polypeptides having the activity.

### Embodiment 2

The method of embodiment 1, wherein said selecting produces an at least 2-fold enrichment in polynucleotides that encode a polypeptide having the activity.

### Embodiment 3

The method of embodiment 2, wherein said selecting produces a degree of enrichment selected from the group consisting of at least: 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold , 10³-fold, 10⁴-fold, 10⁵-fold, 10⁶-fold, 10⁸-fold, and 10⁹-fold enrichment in polynucleotides that encode a polypeptide having the activity.

### Embodiment 4

A method of comparing at least two libraries of polynucleotides with respect to the level of polynucleotides likely to encode functional polypeptides, the method including: (a) providing at least two different libraries of polynucleotides encoding variants of the same polypeptide; (b) for each plurality: (i) producing polypeptides from the polynucleotides; and (ii) determining whether the polypeptides are soluble; and (c) identifying the plurality that has the highest level of soluble polypeptides as the one that contains the highest level of polynucleotides likely to encode functional polypeptides.

### Embodiment 5

The method of embodiment 4, wherein each plurality includes at least some polynucleotides encoding a polypeptide having at least one activity, and the method additionally includes screening the identified plurality for polynucleotides encoding polypeptides having the activity.

### Embodiment 6

The method of any preceding embodiment, wherein the method additionally includes screening the enriched or identified plurality for polynucleotides encoding polypeptides having an activity above a predetermined level.

### Embodiment 7

The method of any preceding embodiment, wherein the producing of (b) includes expressing the polypeptides in a host cell.

### Embodiment 8

The method of embodiment 7, wherein the polypeptides are expressed as fusion proteins in a host cell, wherein the fusion protein also includes a solubility reporter portion.

### Embodiment 9

The method of embodiment 8, wherein the host cell expresses a complementation polypeptide that is capable of binding to the solubility reporter portion of fusion proteins to produce a detectable protein complex.

### Embodiment 10

The method of embodiment 9, wherein the producing of (b) includes: expressing the fusion proteins in the host cell from an expression vector including an inducible or repressible promoter; terminating expression of the fusion proteins and allowing the host cells to rest for a period sufficient to permit degradation or processing of misfolded fusion proteins; after said rest period, expressing the complementation polypeptide from an inducible or repressible promoter.

### Embodiment 11

The method of embodiment 10, wherein determining whether the polypeptides are soluble includes screening the host cells for the detectable protein complex.

### Embodiment 12

The method of embodiment 11, wherein selecting the polynucleotides that encode soluble polypeptides to form an enriched plurality of polynucleotides includes: lysing any host cell(s) including the detectable protein complex; and recovering the polynucleotide encoding the fusion protein.

### Embodiment 13

The method of embodiment 12, wherein the polynucleotide encoding the fusion protein is recovered by nucleic acid amplification.

### Embodiment 14

The method of any of embodiments 1-6, wherein the producing of (b) includes expressing the polypeptides in a reaction mixture including components for in vitro transcription/translation.

### Embodiment 15

The method of embodiment 14, wherein different polypeptides are expressed in separate reaction mixtures including components for in vitro transcription/translation, wherein at least about 20% of the reaction mixtures express one or fewer of said polypeptides (i.e., one polypeptide or no polypeptides) per reaction mixture.

### Embodiment 16

The method of embodiment 15, wherein the separate reaction mixtures comprise aqueous phase droplets in a water-in-oil emulsion.

### Embodiment 17

The method of embodiment 16, wherein the polypeptides are expressed as fusion proteins, wherein each fusion protein includes solubility reporter portion(s) including: a polypeptide attachment tag that is capable of forming a covalent bond with, or otherwise binding to, a polynucleotide of the plurality; and a polypeptide affinity tag.

### Embodiment 18

The method of embodiment 17, wherein the producing of (b) includes: expressing the fusion proteins in the aqueous phase droplets; and permitting each fusion protein that is capable of forming a covalent bond with, or otherwise binding to, a polynucleotide in the aqueous phase droplet to do so.

### Embodiment 19

The method of embodiment 18, wherein determining whether the polypeptides are soluble, and selecting the polynucleotides that encode soluble polypeptides to form an enriched plurality of polynucleotides includes recovering polynucleotides bound to fusions proteins by affinity capture.

### Embodiment 20

The method of any preceding embodiment, wherein the plurality of polynucleotides includes a polynucleotide plurality derived from a sample from a plant or animal or an environmental sample.

### Embodiment 21

A method of screening for chaperones that facilitate protein folding, the method including: (a) expressing a fusion protein in a host cell or in an in vitro reaction mixture, wherein the fusion protein includes a portion that tends to misfold or aggregate, wherein the portion is linked to one or more solubility reporter portion(s), and wherein the host cell or in vitro reaction mixture also includes or produces a potential chaperone; (b) determining whether the fusion protein is soluble; (c) if the fusion protein is soluble, identifying the potential chaperone as one that facilitates folding of the fusion protein.

### Embodiment 22

The method of embodiment 21, wherein the fusion protein is expressed in a plurality of host cells or in vitro reaction mixtures, and different host cells or reaction mixtures, respectively, comprise or produce different potential chaperones, and wherein, when the fusion protein is expressed in in vitro reaction mixtures, at least about 20% of the reaction mixtures comprise or produce one or fewer of said potential chaperones per reaction mixture.

### Embodiment 23

The method of embodiment 22, wherein the potential chaperones are expressed from a polynucleotide plurality selected from a plurality encoding known chaperone polypeptides, a plurality encoding variants of one or more known chaperone polypeptides, a plurality encoding peptides, a plurality derived from a sample from a plant or animal or an environmental sample, or the potential chaperones comprise a small-molecule plurality selected from a plurality of known small-molecule chaperones, a plurality of variants of one or more known small-molecule chaperones, and a plurality of small molecules, wherein each small molecule in the small-molecule plurality is linked to a unique polynucleotide barcode.

### Embodiment 24

The method of embodiment 23, wherein a given potential chaperone is a polypeptide or peptide that is produced in the host cell or in vitro reaction mixture by expressing a polynucleotide encoding the potential chaperone.

### Embodiment 25

The method of any of embodiments 22-24, wherein the fusion protein is expressed in host cells that express a complementation polypeptide that is capable of binding to the solubility reporter portion of fusion proteins to produce a detectable protein complex.

### Embodiment 26

The method of embodiment 25, wherein determining whether the fusion proteins are soluble includes screening the host cells for the detectable protein complex.

### Embodiment 27

The method of embodiment 24, wherein identifying the potential chaperone includes: lysing any host cell(s) including the detectable protein complex; and recovering a polynucleotide encoding the potential chaperone.

### Embodiment 28

The method of embodiment 27, wherein the polynucleotide encoding the potential chaperone is recovered by nucleic acid amplification.

### Embodiment 29

The method of any of embodiments 22-24, wherein the fusion proteins are expressed in separate reaction mixtures that comprise aqueous phase droplets in a water-in-oil emulsion.

### Embodiment 30

The method of embodiment 29, wherein each fusion protein includes solubility reporter portion(s) including: a polypeptide attachment tag that is capable of forming a covalent bond with, or otherwise binding to, a polynucleotide; and a polypeptide affinity tag.

### Embodiment 31

The method of embodiment 30, wherein the method includes permitting each fusion protein that is capable of forming a covalent bond with, or otherwise binding to, a polynucleotide encoding, or bound to, a potential chaperone in the aqueous phase droplet to do so.

### Embodiment 32

The method of embodiment 31, wherein determining whether the fusion protein is soluble includes recovering polynucleotides bound to fusions proteins by affinity capture.

### Embodiment 33

A method of protein domain mapping to identify one or more soluble and/or functional domain(s), the method including: (a) expressing a fusion protein in a host cell or in an in vitro reaction mixture, wherein the fusion protein includes a portion of a protein to be mapped, wherein the portion is linked to one or more solubility reporter portion(s); (b) determining whether the fusion protein is soluble; (c) if the fusion protein is soluble, identifying the portion as one that is a soluble and/or functional domain.

### Embodiment 34

The method of embodiment 33, wherein a plurality of different fusion proteins is expressed in a plurality of host cells or in vitro reaction mixtures, and wherein, when the fusion protein is expressed in in vitro reaction mixtures, at least about 20% of the reaction mixtures comprise or produce one or fewer of said potential chaperones per reaction mixture.

### Embodiment 35

The method of embodiment 34, wherein the plurality of different fusion proteins includes a plurality of different portions of the protein to be mapped, each linked to one or more solubility reporter portion(s).

### Embodiment 36

The method of embodiment 34 or 35, wherein the fusion proteins are expressed in a host cell that expresses a complementation polypeptide that is capable of binding to the solubility reporter portion of soluble fusion proteins to produce a detectable protein complex.

### Embodiment 37

The method of embodiment 36, wherein determining whether the fusion proteins are soluble includes screening the host cells for the detectable protein complex.

### Embodiment 38

The method of embodiment 33, wherein identifying the portion as one that is a soluble and/or functional domain includes: lysing any host cell(s) including the detectable protein complex; and recovering the polynucleotide encoding the fusion protein.

### Embodiment 39

The method of embodiment 38, wherein the polynucleotide encoding the fusion protein is recovered by nucleic acid amplification.

### Embodiment 40

The method of embodiment 34 or 35, wherein the fusion proteins are expressed from polynucleotides encoding the fusion proteins in separate reaction mixtures that comprise aqueous phase droplets in a water-in-oil emulsion.

### Embodiment 41

The method of embodiment 40, wherein each fusion protein includes solubility reporter portion(s) including: a polypeptide attachment tag that is capable of forming a covalent bond with, or otherwise binding to, a polynucleotide encoding the fusion protein; and a polypeptide affinity tag.

### Embodiment 42

The method of embodiment 41, wherein expressing the fusion proteins includes: expressing the fusion proteins in the aqueous phase droplets; and permitting each fusion protein that is capable of forming a covalent bond with, or otherwise binding to, the polynucleotide encoding the fusion protein to do so.

### Embodiment 43

The method of embodiment 42, wherein determining whether the fusion proteins are soluble, and identifying the portion as one that is a soluble and/or functional domain includes recovering polynucleotides bound to fusion proteins by affinity capture.

### BRIEF DESCRIPTION OF THE DRAWINGS

FIG. 1 shows a schematic of a FACS-based library enrichment method. A polynucleotide library fused to a GFP11 tag in an expression vector is transformed into host cells containing a GFP1-10 expression vector (See, Waldo & Cabantous, “Nucleic acid encoding a self-assembling split-fluorescent protein system,” U.S. Pat. No. 7,955,821, which is hereby incorporated by reference for its description of this method). Expression of the fused library proteins is induced (1.). The cells are allowed to rest for 0-2 hours to halt fusion protein expression and to allow for waste processing of misfolded proteins (2.). GFP1-10 expression is separately induced. Positively folded proteins present the GFP11 tag which associate with GFP1-10, resulting in GFP complementation and formation of the fluorophores (3.). Fluorescence-activated cell sorting is used to identify and retain cells expressing positively folding variant proteins (4.) Retained cells are lysed and the polynucleotides encoding positively folding variant proteins are recovered (5.).

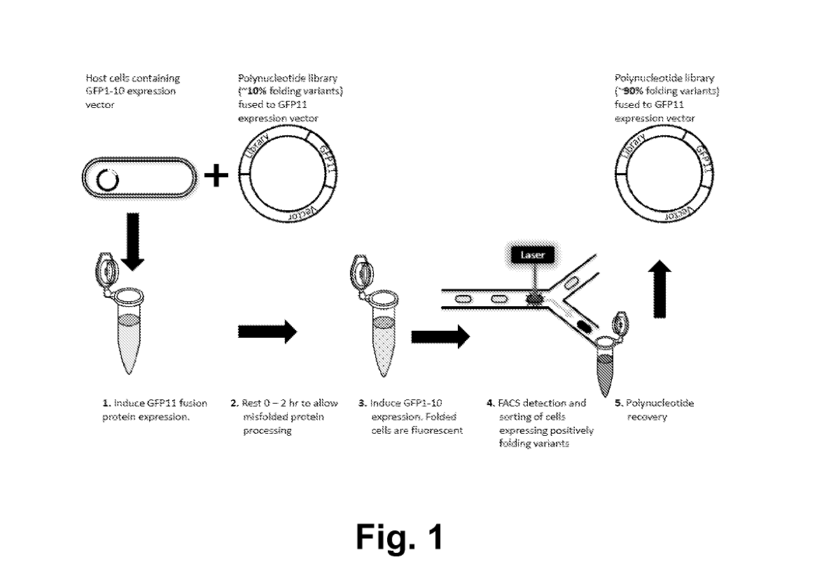

FIG. 2 shows fluorescence data from positive (F+) and negative (NF) folding controls as analyzed by flow cytometry. The histogram plot shows baseline separation between F+ and NF.

FIG. 3 shows a dot plot of fluorescence data from positive (F+) and negative (NF) folding controls and exemplary gating bounds (thick black border) for sorting positively folding proteins.

FIG. 4 shows fluorescence data generated from Thermoascus aurantiacus GH5 wild-type (WT) endoglucanase, insoluble variant (IV) and library generated through error-prone PCR (EPL). See Example 3. The histogram plot shows separation between WT and IV, with the expected overlapping florescence profile of the EPL.

FIG. 5 graphs the percent active clones in a starting library and the enriched libraries generated by sorting and retaining either the highest 5% or 1% fluorescing cells using fluorescence-activated cell sorter. See Example 3.

### DETAILED DESCRIPTION

Unexpectedly, it is feasible to increase the functional density of a polynucleotide library by performing a pre-screen based not on, for example, protein activity or ligand binding, but solely on a test for the expression of soluble protein from each variant. Because protein solubility is an indicator of proper protein folding and proper folding is, in most cases, essential for bioactivity, a method that removes variants that express misfolded proteins will enrich library functional density.

### Definitions

Terms used in the claims and specification are defined as set forth below unless otherwise specified.

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers to any form of DNA or RNA, including, for example, genomic DNA; complementary DNA (cDNA), which is a DNA representation of messenger RNA (mRNA), usually obtained by reverse transcription of mRNA or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

“Specific hybridization” refers to the binding of a polynucleotide to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

In particular embodiments, hybridizations are carried out under stringent hybridization conditions. The phrase “stringent hybridization conditions” generally refers to a temperature in a range from about 5° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) Methods In Enzymology, Vol.152: Guide To Molecular Cloning Techniques, San Diego: Academic Press, Inc. and Sambrook et al. (1989) Molecular Cloning: A Laboratory Manual, 2nd Ed., Vols. 1-3, Cold Spring Harbor Laboratory), both incorporated herein by reference). As indicated by standard references, a simple estimate of the Tₘ value may be calculated by the equation: Tₘ=81.5+0.41(% G+C), when a nucleic acid is in aqueous solution at 1 M NaCl (see, e.g., Anderson and Young, Quantitative Filter Hybridization in Nucleic Acid Hybridization (1985)). The melting temperature of a hybrid (and thus the conditions for stringent hybridization) is affected by various factors such as the length and nature (DNA, RNA, base composition) of the hybridizing polynucleotide and nature of the target polynucleotide (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH 7.

The terms “polypeptide, “oligopeptide”, and “peptide” are used herein to refer a polymer of amino acids, and unless otherwise limited, include atypical amino acids that can function in a similar manner to naturally occurring amino acids. The term “polypeptide” is understood as a generic term, although when used in conjunction with the term(s) “oligopeptide(s)” and/or “peptide(s),” those of skill in the art readily appreciate that, in this context, polypeptides are typically longer than oligopeptides (e.g., longer than about 20 amino acids), which are typically longer than peptides (e.g., longer than about 5 amino amino acids).

The term “amino acid” includes naturally occurring L-amino acids, unless otherwise specifically indicated. The term “amino acid” includes D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid”.

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2, 3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3- (and 4-) hydoxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

A polypeptide is said to have at least one activity if that polypeptide can carry out at least one function of interest. Exemplary functions include the ability to bind (specifically or non-specifically to) another entity, which can, but need not be, another polypeptide; the ability to modulate the function (e.g., enhance, synergize, inhibit) of itself or another entity, which can, but need not be another polypeptide; enzymatic activity; the ability to serve as a substrate; immunogenic activity (i.e., the ability to elicit an immune response).

Polynucleotides and polypeptides are said to be “different” if they differ in structure, e.g., nucleotide sequence for polynucleotides and amino acid sequence for polypeptides.

As used herein, polypeptides or fusion proteins are “soluble” if they are recoverable from the soluble fraction of lysed host cells.

As used herein, “positively folded” describes polypeptides or polypeptide domains that are sufficiently properly folded that they are soluble. “Positive folding” does not necessarily imply complete proper folding.

The term “solubility reporter portion” is used herein to refer to any portion of a fusion protein that provides a function important in a solubility assay.

The term “polypeptide affinity tag” refers to any amino acid sequence that can function, or can conveniently be modified to function, directly or indirectly, as an affinity tag to facilitate affinity purification. For example, a poly(His) tag represents a direct affinity tag when affinity purification is based on binding of the poly(His) to a metal matrix. An amino acid sequence that is the epitope for a biotinylated antibody represents an indirect affinity tag when affinity purification is based on the binding of the biotinylated antibody to the epitope, followed by binding of the resulting complex to avidin.

As used herein, the term “recovering polynucleotides” does not require physical recovery of particular polynucleotides. Polynucleotides can be “recovered,” for example, by producing one or more copies of the polynucleotide, e.g., by amplification, DNA sequencing, etc.

### Methods

### Library Enrichment Based on Solubility of Encoded Polypeptides

### In General

Described herein are methods of enriching a plurality of polynucleotides, such as a polynucleotide library, for polynucleotides likely to encode functional polypeptides. The enriched plurality of polynucleotides can then be screened for polynucleotides encoding polypeptides having a desired activity. These methods improve the efficiency of library screening by eliminating the need to screen improperly folded polypeptide variants. The methods entail providing a plurality of polynucleotides encoding variants of a polypeptide, wherein at least some of the polynucleotides encode a polypeptide having at least one activity. Polypeptides are produced from the polynucleotides, followed by a determination of whether the polypeptides are soluble, which serves as an indicator of positive folding. The polynucleotides that encode soluble polypeptides are selected to form the enriched plurality of polypeptides that can then be screened for activity using any conventional screening method.

In certain embodiments, the methods described herein are useful for screening libraries of polynucleotides. In particular embodiments, the methods employ libraries having at least about: 10², 10³, 10⁴, 10⁵, 10⁶, 10⁷, 10⁸, 10⁹, 10¹⁰, 10¹¹, and 10¹² different polynucleotides. Generally, the size of the library will be less than about 10¹⁵ different polynucleotides. Polynucleotide library sizes can also fall within any range bounded by any of these values, e.g., 10²-10¹⁵, 10³-10¹², 10³-10¹⁰, 10³-10⁹, 10³-10⁸, 10³-10⁷, 10³-10⁶, etc.

Pluralities of polynucleotides useful in the methods described herein can be obtained from any source and/or be generated in any manner. In various embodiments, they can be derived from a sample from a plant or animal or from an environment sample. For example, pluralities of polynucleotides can be derived from bacteria, protozoa, fungi (e.g., yeast, filamentous fungi), viruses, organelles, as well higher organisms such as plants or animals, particularly mammals, and more particularly primates, and even more particularly humans. Libraries of polynucleotides can be created in any of a variety of different ways that are well known to those of skill in the art. In particular, pools of naturally occurring polynucleotides can be cloned from genomic DNA or cDNA (Sambrook et al., 1989, Molecular Cloning: A Laboratory Manual, 2nd Ed., Vols. 1-3, Cold Spring Harbor Laboratory); for example, phage antibody libraries, made by PCR amplification repertoires of antibody genes from immunized or unimmunized donors have proved very effective sources of functional antibody fragments (Winter et al., Annu Rev Immunol. 1994;12:433-55; Hoogenboom, Trends Biotechnol. 1997 Feb;15(2):62-70). Libraries of genes can also be made by encoding all (see for example Smith, Science 1985 Jun 14;228(4705):1315-7; Parmley and Smith, Gene 1988 Dec 20;73(2):305-18) or part of genes (see for example Lowman et al., Gene 1988 Dec 20;73(2):305-18) or pools of genes (see for example Nissim et al., Gene 1988 Dec 20;73(2):305-18) by a randomized or doped oligonucleotide synthesis. Libraries can also be made by introducing mutations into a polynucleotide or pool of polynucleotides randomly by a variety of techniques in vivo, including; using mutator strains, of bacteria such as E. coli mutD5 (Liao et al., Proc Natl Acad Sci U S A. 1986 Feb;83(3):576-80; Yamagishi et al., Protein Eng. 1990 Aug;3(8):713-9; Low et al., 1 J Mol Biol. 1996 Jul 19;260(3):359-68); using the antibody hypermutation system of B-lymphocytes (Yelamos et al., Nature 1995 Jul 20;376(6537):225-9). Random mutations can also be introduced both in vivo and in vitro by chemical mutagens, and ionizing or UV irradiation (see Friedberg et al., Philos Trans R Soc Lond B Biol Sci. 1995 Jan 30;347(1319):63-8), or incorporation of mutagenic base analogues (Freese, Proc Natl Acad Sci U S A. 1959 Apr;45(4):622-33; Zaccolo et al., J Mol Biol. 1996 Feb 2;255(4):589-603). Random mutations can also be introduced into genes in vitro during polymerization for example by using error-prone polymerases (Leung et al., Technique 1989 1: 11-15). Further diversification can be introduced by using homologous recombination either in vivo (see Kowalczykowski et al., Microbiol Rev. 1994 Sep;58(3):401-65) or in vitro (Stemmer, Proc Natl Acad Sci USA. 1994 Oct 25;91(22):10747-51; Stemmer, Nature 1994 Aug 4;370(6488):389-91). Libraries of complete or partial genes can also be chemically synthesized from sequence databases or computationally predicted sequences.

Polypeptides are most conveniently produced from a plurality of polynucleotides using a suitable expression vector. Therefore, polynucleotide libraries useful in the methods disclosed herein are typically in, or can be inserted into, an expression vector that functions in the intended host cell or in vitro transcription/translation system. Expression vectors can include suitable regulatory sequences, such as those required for efficient expression of the gene product, for example promoters, enhancers, translational initiation sequences, polyadenylation sequences, splice sites and the like. A wide variety of expression vectors and host cells are available and known those of skill in the art.

Expressed polypeptide solubility can be determined by any convenient available method. The method is one that permits, and preferably facilitates, the recovery of a polynucleotide that encodes a polypeptide determined to be soluble. Preferably, the method is one that facilitates solubility assay and polynucleotide recovery in a high-throughput manner, e.g., where all members of a polynucleotide library can be screened for ability to express a soluble polypeptide in a single assay. For example, useful high-throughput systems include those in which each polypeptide is expressed so as to be “packaged” with, or linked with, the encoding polynucleotide, e.g., in a cell, particle, droplet, or like, where each “package” (e.g., cell) or linked complex can be separately analyzed for a signal that indicates expression of a soluble polypeptide and then sorted based on this signal. Signal detection and sorting can be carried out using, e.g., a fluorescence-activated cell sorter (FACS) or a microfluidic device.

Polynucleotides can be packaged with their expressed polypeptides by expression in vivo, i.e., in a host cell. Polynucleotides can also be packaged with their expressed polypeptides by expression in vitro, e.g., in a reaction mixture including components for in vitro transcription/translation. In various embodiments, at least about: 20%, 30%, 40%, 50%, 60%, 70%, 80%, or 90% of the reaction mixtures express one or fewer types of polypeptides (i.e., one polypeptide or no polypeptides) per reaction mixture; the percentage of reaction mixtures that express one or fewer polypeptides per reaction mixture can also fall within any range bounded by these values. The reaction mixtures can, for example, be aqueous phase droplets in a water-in-oil emulsion.

In particular embodiments, the polynucleotides of a plurality are each fused to a polynucleotide encoding a solubility reporter portion. In such embodiments, production of the polypeptides for solubility determination can entail production of the fusion proteins, on which the solubility determination is carried out. Positive folding in the portion of the fusion that corresponds to the polypeptide under analysis is typically associated with a functional solubility reporter.

In some embodiments, the host cell or in vitro transcription/translation system expresses a complementation polypeptide that is capable of binding to the solubility reporter portion of fusion proteins to produce a detectable protein complex. For example, the fusion proteins can be expressed from an expression vector including an inducible or repressible promoter, which permits termination of expression of the fusion proteins, followed by a rest period to permit degradation or processing of misfolded fusion proteins. After the rest period, the complementation polypeptide can be expressed from a different inducible or repressible promoter. The complementation polypeptide forms a detectable protein complex with any positively folded fusion proteins, and the host cells or the in vitro transcription/translation reaction mixtures can be screened for the protein complex.

In some embodiments, an in vitro transcription/translation system, e.g., in aqueous droplets in an emulsion, expresses a fusion protein including a solubility reporter portion that includes a polypeptide attachment tag that is capable of forming a covalent bond with, or otherwise binding to, a polynucleotide of the plurality. The fusion protein also includes a polypeptide affinity tag. In such embodiments, fusion proteins can be expressed under conditions that permit each fusion protein that is capable of forming a covalent bond with, or otherwise binding to, a polynucleotide in the aqueous phase droplet to do so. The in vitro transcription/translation reaction mixtures can be screened for soluble fusion proteins by affinity capture.

In certain embodiments, a plurality of polynucleotides that encode soluble polypeptides is selected to form an enriched plurality of polynucleotides. Polynucleotides packaged with or linked to soluble polypeptides can be recovered individually or in a pool. Recovered polynucleotides can serve as an enriched plurality of polynucleotides, which can, optionally, be subjected to further screening. Where solubility screening is based on the detection of protein complex in a host cell, polypeptides can be recovered by lysing any host cell(s) comprising the detectable protein complex, and recovering the polynucleotide encoding the fusion protein. When solubility screening is based on affinity capture of a fusion protein linked to the polynucleotide that encodes it, the screening step yields recovered polynucleotides. In either case, polynucleotide recovery can also entail nucleic acid amplification, e.g., with primers that anneal to vector regions flanking the polynucleotide.

The enriched plurality of polynucleotides can be subjected to additional screening to identify one or more polynucleotides having, or likely to have, a desired activity. Screening can be carried out by expressing the encoded polypeptides or a portion thereof and then screening for a desired characteristic, such as protein binding (either specific or non-specific), enzymatic activity, synergistic or inhibitory activity, desorption, adsorption, etc. Alternatively, or in addition, screening can entail sequencing the polynucleotides and identifying polynucleotides having a sequence or sequence motif of interest, altered sequence frequency, selected sequence identities, or counter-selected sequence identities.

In some embodiments, the enriched plurality of polypeptides is screened for polynucleotides encoding polypeptides having an activity above a predetermined level. In certain embodiments, an activity screen is carried out to identify polypeptides having an activity level that is higher or lower than a wild-type activity level or than a “starting” activity level, e.g., that is characteristic of a polypeptide prior to mutagenesis of the corresponding polynucleotide to produce a polynucleotide library. In various embodiments, the activity screen can identify polypeptides having an activity level that differs from a wild-type or a starting activity level by at least: 2%, 5%, 10%, 15%, 20%, 25%, 30%, 35%, 40%, 45%, 50%, 55%, 60%, 65%, 70%, 75%, 80%, 85%, 90%, 95%, 2-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 500-fold, 1,000-fold 5,000-fold, 10,000-fold. In some embodiments, the difference in activity level falls within a range bounded by any of these values, e.g., 2-fold to 10,000-fold, 2-fold to 100-fold, 5-fold to 95-fold, 10-fold to 90-fold, 15-fold to 85-fold, 20-fold to 80-fold, 25-fold to 75-fold, 30-fold to 75-fold, etc. Such a screen can be carried out to recover polynucleotides encoding polypeptides having a desired activity.

In particular embodiments, the selection produces an enriched plurality of polynucleotides that has at least 2-fold enrichment in polynucleotides that encode a polypeptide having the activity. In various embodiments, the degree of enrichment is at least: 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold , 10³-fold, 10⁴-fold, 10⁵-fold, 10⁶-fold, 10⁸-fold, or 10⁹-fold. In some embodiments, the degree of enrichment falls within a range bounded by any of these values, e.g., 2-fold to 100-fold, 5-fold to 95-fold, 10-fold to 90-fold, 15-fold to 85-fold, 20-fold to 80-fold, 25-fold to 75-fold, 30-fold to 75-fold, 100-fold to 10⁹-fold, 10³-fold to 10⁸-fold, etc.

### A. Library Enrichment by Fusion of the Library to a Protein Solubility Reporter—In Vivo System

In particular embodiments, an in vivo enrichment method entails fusion of the library members to a protein solubility reporter, followed by selection of polynucleotides encoding soluble fusion proteins. In an illustrative embodiment, each member of a polynucleotide library is fused via a linker to the polynucleotide encoding an 11 beta-strand polypeptide solubility reporter (Waldo & Cabantous, “Nucleic acid encoding a self-assembling split-fluorescent protein system,” U.S. Pat. No. 7,955,821, which is hereby incorporated by reference for this description) in a suitable expression vector (e.g., T7 promoter-based vector).

The fusion protein library is transformed into a plurality of Escherichia coli containing a tetracycline-inducible expression vector encoding a polypeptide corresponding to beta-strands 1-10 of the fluorescent protein (Waldo & Cabantous, supra, which is hereby incorporated by reference for this description) which is capable of self-complementation with the 11 beta-strand polypeptide fusion protein.

The fusion protein library is expressed in a plurality of Escherichia coli using the T7 inducible expression system (U.S. Pat. No. 5,693,489 which is hereby incorporated by reference for this description) mediated by the addition of isopropyl β-D-1-thiogalactopyranoside (IPTG).

The Escherichia coli are washed to remove the IPTG and halt fusion protein expression.

The Escherichia coli are allowed to rest for 0-2 hours to allow for folding or misfolding and degradation/processing of the fusion protein library.

Expression of a polypeptide corresponding to beta-strands 1-10 of the fluorescent protein is induced by the addition of tetracycline or a tetracycline derivative (e.g., anhydrotetracycline (aTc)).

Positively folding fusion protein variants are capable of self-complementation and reconstitution of the fluorescent protein whereas misfolded fusion protein variants do not reconstitute the fluorescent proteins.

Positively folding fusion protein variants are detected, selected, and retained by using fluorescence-activated cell sorting (FACS). Misfolding fusion protein variants may also be retained separately as well.

The retained Escherichia coli are lysed and the polynucleotide encoding the variant is recovered.

### B. Other Embodiments

The illustrative enrichment method described above can be carried using a different folding reporter system. Any system with a detectable output that is mediated by proper protein folding or protein solubility can be employed. For example, folding reporter GFP (Waldo et al., 1999, Nat. Biotechnol. 17:691-695, which is hereby incorporated by reference for this description; U.S. Pat. No. 6,448,087, which is hereby incorporated by reference for this description), any GFP, GFP-like fluorescent proteins, chemiluminescent, and chromophoric proteins may be employed in the practice of the methods described herein. Additionally, any other split-reporter protein systems, employing any number of proteins with a detectable phenotype, such as the enzyme beta lactamase, beta galactosidase (Ullmann, Jacob et al., J Mol Biol. 1967 Mar 14;24(2):339-43; Welply, Fowler et al., J Biol Chem. 1981 Jul 10;256(13):6804-10; Worrall and Goss, Aust J Biotechnol. 1989 Jan;3(1):28-32; Jappelli, Luzzago et al., J Mol Biol. 1992 Sep 20;227(2):532-43; Rossi, Blakely et al., Methods Enzymol. 2000;328:231-51; Wigley, Stidham et al., Nat Biotechnol. 2001 Feb;19(2):131-6; Lopes Ferreira and Alix, J Bacteriol. 2002 Dec;184(24):7047-54), dihydrofolate reductase (Gegg, Bowers et al., Protein Sci. 1997 Sep;6(9):1885-92; Iwakura and Nakamura, Protein Eng. 1998 Aug;11(8):707-13; Pelletier, Campbell-Valois et al., Proc Natl Acad Sci USA. 1998 Oct 13;95(21):12141-6; Pelletier, Arndt et al., Nat Biotechnol. 1999 Jul;17(7):683-90; Iwakura, Nakamura et al., Nat Struct Biol. 2000 Jul;7(7):580-5; Smith and Matthews, Protein Sci. 2001 Jan;10(1):116-28; Arai, Maki et al., J Mol Biol. 2003 Apr 18;328(1):273-88), or chloramphenicol resistance protein may be used, or rescue of an auxotrophic phenotype, or protease complementation. Additionally, fluorescence resonance energy transfer may be used to determine if two fluorophores attached to or associated with the polypeptide are within a certain distance of each other or are stably fixed at a certain distance as an indicator of positive folding.

Different expression hosts can be used in the illustrative enrichment embodiment described above, e.g., Saccharomyces cerevisiae, Pichia pastoris, Chinese hamster ovary (CHO) cells, and the like, as can cell-free in vitro expression systems.

Different induction systems can be used in the illustrative enrichment embodiment described above, e.g., any controllable protein induction system (e.g., arabinose-inducible araBAD promoter, temperature-inducible promoters, stress-inducible promoters).

The rest time can be as long as 24 hours or more depending on the host cell and protein of interest. Exemplary rest times range from 0 hours (i.e., no rest time) to 120 hours or more, e.g., 2 hours, 4 hours, 6 hours, 8 hours, 10 hours, 12 hours, 14 hours, 16 hours, 18 hours, 20 hours, 22 hours, or any time falling within any range bounded by these values, e.g., 2-24 hours, 4-18 hours, 6-18 hours, etc.

Different detection and selection systems can be used in the illustrative enrichment embodiment described above. For example, the detection systems may be colorimetric, fluorescent, chemiluminescent, affinity, electrical, or chemical depending on the reporter protein used. Host cells or droplets encapsulating in vitro-expression systems may be selected by any number of sorting techniques (e.g., acoustic, dielectrophoretic, electric charge) or by affinity interaction with, for example, antibodies or by self-selecting means such as, e.g., cell viability, aggregation, sedimentation, or buoyancy.

### C. Library Enrichment by Fusion of the Library to a Protein Solubility Reporter—Folding-Based Self-Selecting in Vitro System

In particular embodiments, an in vitro enrichment method entails fusion of the library members to a protein solubility reporter, followed by selection of polynucleotides encoding soluble fusion proteins. In an illustrative embodiment, each member of a polynucleotide library is fused to an inducible promoter (e.g., T7), a polynucleotide encoding a polypeptide “attachment tag” (e.g., SNAP-, CLIP-, ACP- and MCP-tags (New England BioLabs Inc., USA)) capable of forming a bond or affinity with a polynucleotide or polynucleotide derivative, and to a polynucleotide encoding a polypeptide “affinity tag” (e.g., FLAG-tag: DYKDDDDK). The library may be synthesized or otherwise derivatized to enable bond or affinity formation between the polynucleotide and the attachment tag (e.g., benzylguanine-modified DNA for use with the SNAP-tag).

The fusion protein library is dispersed in an emulsion containing an in vitro transcription/translation reagent such that on average one or fewer library fusion elements is expressed within each droplet compartment.

Positively folding fusion protein variants are capable of bond or affinity interaction with the encoding polynucleotide creating a polynucleotide-attachment tag-affinity tag complex, whereas misfolded fusion protein variants do not form a bond or affinity with the encoding polynucleotide.

The emulsion is then broken and polynucleotide variants encoding positively folding fusion proteins are recovered by affinity capture of the affinity tag (e.g., using biotinylated M5 anti-FLAG (Sigma-Aldrich, USA) according to Sepp et al., FEBS 2002:532, 455-458.) Polynucleotide variants encoding misfolding fusion proteins may also be recovered by subtractive removal of positively folding fusion proteins as above.

### Library Analysis

Polynucleotide libraries are generated using a variety of means (e.g., error-prone PCR, site-directed mutagenesis, computational design and synthesis, and molecular breeding (Stemmer, U.S. Pat. No. 5,605,793)). The researcher must make certain guesses and assumptions when creating a polynucleotide library. Because of this uncertainty, multiple libraries are typically generated and tested. Variations on any of the above-described methods may be used to analyze the ratio of folding and misfolding variants in the generated libraries without sorting or recovery of the polynucleotides. This analysis is useful, for example, in understanding how sensitive a particular protein is to mutational change, in analyzing the effect of different mutagenic techniques, in probing the intrinsic folding stability of a protein, in probing specific sites in a protein that affect stability, in tuning computational models used for library generation, and in investigating the compatibility of combinatorial elements used in molecular breeding.

Accordingly, described herein are methods of comparing at least two pluralities of polynucleotides, e.g., two polynucleotide libraries, with respect to the level of polynucleotides likely to encode functional polypeptides. The methods entail providing at least two different pluralities of polynucleotides encoding variants of the same polypeptide. For each plurality, polypeptides are produced from the polynucleotides. For each plurality, the polypeptides are screened to determine whether they are positively folded and/or soluble. The plurality that has the highest level of positively folded and/or soluble polypeptides is identified as the one that contains the highest level of polynucleotides likely to encode functional polypeptides. In some embodiments, each plurality includes at least some polynucleotides encoding a polypeptide having at least one activity, and the method additionally entails screening the plurality identified as the one likely to encode more functional polypeptides, wherein the screening is carried out to identify polynucleotides encoding polypeptides having the activity.

In some embodiments, the identified plurality of polypeptides is screened for polynucleotides encoding polypeptides having an activity above a predetermined level. In certain embodiments, an activity screen is carried out to identify polypeptides having an activity level that is higher or lower than a wild-type activity level or than a “starting” activity level, e.g., that is characteristic of a polypeptide prior to mutagenesis of the corresponding polynucleotide to produce a polynucleotide library. In various embodiments, the activity screen can identify polypeptides having a an activity level that differs from a wild-type or a starting activity level by at least: 2%, 5%, 10%, 15%, 20%, 25%, 30%, 35%, 40%, 45%, 50%, 55%, 60%, 65%, 70%, 75%, 80%, 85%, 90%, 95%, 2-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 500-fold, 1,000-fold 5,000-fold, 10,000-fold. In some embodiments, the difference in activity level falls within a range bounded by any of these values, e.g., 2-fold to 10,000-fold, 2-fold to 100-fold, 5-fold to 95-fold, 10-fold to 90-fold, 15-fold to 85-fold, 20-fold to 80-fold, 25-fold to 75-fold, 30-fold to 75-fold, etc. Such a screen can be carried out to recover polynucleotides encoding polypeptides having a desired activity.

Screening for Chaperones that Facilitate Protein Folding

Additionally described herein are methods of screening for chaperones that facilitate protein folding. In particular embodiments, such a method entails expressing a fusion protein in a host cell or in an in vitro reaction mixture, e.g., as described above, wherein the fusion protein includes a portion that tends to misfold or aggregate, wherein this portion is linked to one or more solubility reporter portion(s), and wherein the host cell or in vitro reaction mixture also includes or produces a potential chaperone. A solubility determination is carried out, e.g., as described above. If the fusion protein is determined to be soluble, the potential chaperone in the same host cell or in vitro reaction mixture is identified as one that facilitates folding of the fusion protein. A plurality of chaperones can be screened for the ability to facilitate folding of a given fusion protein by expressing the fusion protein in a plurality of host cells or in vitro reaction mixture, wherein different host cells or reaction mixtures, respectively, include or produce different potential chaperones.

More than one potential type of chaperone can be expressed per host cell or reaction mixture, but when investigating the effects of individual potential chaperones on folding, it is desirable to adjust conditions so that one potential chaperone is present in each host cell or reaction mixture. In in vivo embodiments, one can, e.g., use an expression vector with a selectable marker to ensure that all transformed cells express a chaperone, and, if each vector encodes a single chaperone, each transformed cell will typically express that chaperone. In various in vitro embodiments, at least about: 20%, 30%, 40%, 50%, 60%, 70%, 80%, or 90% of reaction mixtures include one or fewer potential chaperones per reaction mixture; the percentage of reaction mixtures that include one or fewer potential chaperones per reaction mixture can also fall within any range bounded by these values.

In embodiments in which the potential chaperones are expressed in host cells or in reaction mixtures, the potential chaperones can be expressed from a plurality of polynucleotides, such as a plurality encoding known chaperone polypeptides (including known chaperone oligopeptides or shorter peptides), a plurality encoding variants of one or more known chaperone polypeptides, a plurality encoding peptides (e.g., short random sequences of amino acids), or a plurality derived from a sample from a plant or animal or an environmental sample. The plurality of polynucleotides can be a polynucleotide library, e.g., as described above. Known polypeptide chaperones can be from any species, e.g., bacteria, mammals, primates, and humans, and include heat shock proteins (e.g., Hsp60, Hsp70, Hsp90, or Hsp100), foldases (e.g., the GroEL/GroES or the DnaK/DnaJ/GrpE system), holdases (DnaJ or Hsp33), and chaperonins. Human polypeptide chaperones include those found in the endoplasmic reticulum, such as general chaperones (e.g., BiP, GRP94, GRP170), lectin chaperones (e.g., calnexin and calreticulin), non-classical molecular chaperones (e.g., HSP47 and ERp29), and folding chaperones, such as protein disulfide isomerase (PDI), peptidyl prolyl cis-trans-isomerase (PPI), and ERp57.

Alternatively, or in addition, the potential chaperones can include a plurality of small molecules, such as known small-molecule chaperones, variants of one or more known small-molecule chaperones. In some embodiments, each small molecule in the small-molecule plurality is linked to a unique polynucleotide barcode, which permits convenient identification of any chaperones that facilitate folding. More specifically, the use of the polynucleotide barcode permits identification of chaperone identity by nucleic acid amplification. Known small-molecule chaperones include dimethyl sulfoxide (DMSO), trimethylamine n-oxide (TMAO), polyhydric alcohols including glycerol, arabitol, mannitol, and sorbitol, iminosugars (e.g., DGJ or DGJNAc), non-iminosugar glucocerebrosidase inhibitors, methylamines including betaine, glycerophosphorylcholine, sarcosine, and trimethylamine N-oxide.

Alternatively, or in addition, polynucleotides themselves can serve as chaperones directly, i.e., without translation into polypeptides. A plurality of polynucleotides can be tested for this activity used the methods described above, e.g., by introducing polynucleotides that cannot be translated into polypeptides into host cells or reaction mixtures.

In illustrative embodiments, a polynucleotide encoding a misfolding or aggregating protein (e.g., the causative proteins in Alzheimer's disease, Parkinson's disease, Huntington's disease, and other amyloidogenic diseases including type 2 diabetes, inherited cataracts, some forms of atherosclerosis, hemodialysis-related disorders, and short-chain amyloidosis, among many others) is fused to the solubility reporter by any of the means A., B., or C. above.

The fusion protein is expressed in the presence of or sequentially with a plurality of chaperones (a “chaperone library”). The chaperones may be, for example, a library of peptides (e.g., a library containing all possible permutations of 12-24 amino acids), a library of chaperone polypeptides and/or chaperone polypeptide variants, a library of chemical chaperones (e.g., a therapeutic small molecule library). The chaperones are either expressed from a polynucleotide or, in the case of small molecules, have a linked nucleic acid barcode.

Positively folding cells or compartments are selected by any of the means A., B., or C. above

The identity of the chaperone that assists in positive folding is determined by, for example, recovery and sequencing of the polynucleotide encoding the chaperone peptide or polypeptide or the sequencing of a polynucleotide “barcode” that identifies the chemical chaperone. For a description of nucleic acid barcoding of small-molecule libraries, see Clark M a, Acharya R a, Arico-Muendel CC, et al., Design, synthesis and selection of DNA-encoded small-molecule libraries. Nature chemical biology. 2009;5(9):647-54. Available at: www.ncbi.nlm.nih.gov/pubmed/19648931.

### Protein Domain Mapping to Determine Soluble or Functional Subunits

Proteins are composed of domains that are often expressible as separate soluble units. Identification of protein domains plays a central role in protein structure-function analysis, protein crystallization, and the identification sites responsible for polynucleotide, protein, and small-molecule binding.

Accordingly, described herein is a method of protein domain mapping to identify one or more soluble and/or functional domain(s). In certain embodiments, the method entails expressing a fusion protein in a host cell or in an in vitro reaction mixture, wherein the fusion protein includes a portion of a protein to be mapped, and that portion is linked to one or more solubility reporter portion(s). A solubility determination is carried out on the fusion protein. If the fusion protein is soluble, the portion of the protein being mapped can be identified as a soluble and/or functional domain. In particular, since solubility is correlated with positive folding, one can conclude, based on solubility that the portion of the protein being mapped is capable of positive folding and therefore likely functional.

Typically, a series of protein domains are analyzed, which can be achieved by analyzing a plurality of different fusion proteins that includes a plurality of different portions of the protein to be mapped, each linked to one or more solubility reporter portion(s). Thus, in some embodiments, a plurality of different fusion proteins is expressed in a plurality of host cells or in vitro reaction mixtures. In particular embodiments, conditions are be adjusted so that one type of fusion protein is present in each host cell or reaction mixture. In various in vitro embodiments, at least about: 20%, 30%, 40%, 50%, 60%, 70%, 80%, or 90% reaction mixtures include one or fewer fusion proteins per reaction mixture; the percentage of reaction mixtures that include one or fewer potential chaperones reaction mixture can also fall within any range bounded by these values.

In illustrative embodiments, a polynucleotide encoding a random fragmentation or terminal deletion of a polypeptide of interest is fused to the solubility reporter by any of the means A., B., and C. above.

Positively folding domains are selected by any of the means A., B., and C. above.

The recovered polynucleotide fragments or terminal deletions are sequenced and computationally aligned to the reference sequence of the polypeptide of interest to map the extent of soluble domains. Alternatively, hybridization may be used to physically map the recovered polynucleotide fragments or terminal deletions. In some embodiments, for example, the recovered polynucleotide fragments or terminal deletions can be specifically hybridized to one or more references polynucleotides to map the extent of soluble domains.

### Bioprospecting

It is advantageous to isolate unknown proteins from environmental samples, tissue samples, or other isolated biological material that are expressible and perhaps functional in a heterologous host (e.g., fungal cellulases that are expressible in Escherichia coli, human proteases that are expressible in Escherichia coli, thioesterases that are expressible in Saccharomyces cerevisiae, polypeptide chaperones that mediate protein folding in CHO cells).

A polynucleotide library derived from an environmental sample, tissue sample, or other isolated biological material can be used in any of the methods A., B., and C. above, as well as in screening for chaperones that facilitate folding. The in vivo screening methods described above are, in particular, useful for identifying proteins that are expressible and functional in a specific host of interest. In this case, screening can be carried out in a cell from that specific host.

All publications, patents, and patent applications cited herein are hereby incorporated by reference in their entirety for all purposes.

### EXAMPLES

Various aspects of the invention are further described and illustrated by way of the several examples which follow, none of which are intended to limit the scope of the invention.

### Example 1

Library Cloning into pTET GFP 11 Vector

A cellulase gene library was cloned into the Ncol and BamHI restriction sites of the pTET GFP 11 vector (Cabantous & Waldo, In vivo and in vitro protein solubility assays using split GFP, Nat. Meth. 3:845-54 (2006), which is hereby incorporated by reference for this description). The ligation reaction was purified using a spin column kit (QIAGEN Inc., Valencia, Calif.). The DNA was quantitated using absorption at 260 nm (Nanodrop, Thermo, Wilmington, Del.), and 50-200 ng in a maximum of 5 μL volume was used for the transformation of ultracompetent Escherichia coli (XL10-Gold Ultracompetent Cells or ElectroTen-Blue® Cells, Agilent Technologies, Santa Clara, Calif.) according to the manufacturer's protocol, which included outgrowth of the transformed cells for 75 minutes at 37° C. in 1 mL rich liquid media, such as SOC, without selection.

### Supercoil Library Preparation

After outgrowth, the outgrown cells were added to 200 mL of Luria-Bertani (LB) liquid media containing 50 μg/mL spectinomycin in a 0.5 L flask (UltraYield, Thomson Instrument, Oceanside, Calif.) and grown at 37° C. for 12-16 hours to allow transformed cells to become more numerous than untransformed cells. Cells were harvested by centrifugation and library plasmid purified with a commercial midi-prep kit (Macherey Nagel, Bethlehem, Pa.) and quantitated by absorbance at 260 nm. A small portion of the outgrown cells were reserved for determining transformation efficiency and effective library size.

### Host Cell Transformation

50-200 ng of purified plasmid DNA in a maximum of 5 μL, preferably 1 μL, was used to transform electrocompetent Escherichia coli cells that had been previously prepared to contain pET GFP 1-10 (Cabantous & Waldo, supra, which is hereby incorporated by reference for this description). Transformation by electroporation was performed in 1-mm gap electrocuvettes using the EC1 program on a MicroPluser Electroporator (Bio-Rad Laboratories, Inc., Hercules, Calif.). Transformed cells were outgrown in 1 mL of SOC media without selection for 75 minutes at 37° C.

The transformed, outgrown cells now containing both plasmids were used to inoculate 200 mL of liquid LB containing 33 μg/mL kanamycin (LB-K/S) and 75 μg spectinomycin and grown for 12-16 hours at 37° C. in a 0.5 L flask (UltraYield, Thomson Instrument, Oceanside, Calif.). A portion of the culture was then diluted to an optical density (OD) of 0.02, measured at 600 nm, in fresh LB-K/S and grown at 37° C. to OD 0.3.

### Measuring Transformation Efficiency and Effective Library Size

A small portion of the transformed cells was reserved, and two dilutions (1:500 and 1:5000) were plated onto Luria-Bertani (LB) Agar containing 50 μg/mL spectinomycin. After overnight incubation, colonies from both plates were counted to determine the transformation efficiency and effective library size, which were typically 1-5×10⁶.

### Sequential Induction of Fusion Library and Folding Reporter

Cells at OD 0.3 were quickly transferred in 0.5-mL aliquots to a 96-well culture block (#780285, Greiner BioOne, Monroe, N.C.), and induced by the addition of anhydrotetracycline (aTc) at a concentration of 30-600 ng/mL. Cells were incubated with shaking at 1,000 rpm at 37° C. in a microplate shaker with a 3-mm orbit (VWR Symphony, Radnor, Pa.). After 1-3 hours, preferably 2 hours, cells were pelleted by centrifugation, and the aTc-containing media removed and replaced with fresh LB-K/S. The cells were returned to the incubator for 45-180 minutes, preferably 120 minutes, to rest, which allowed misfolded protein variants to be processed out of the soluble-fraction of the cytoplasm.

The second induction was begun at the end of rest by addition of isopropyl □. D-1-thioglactopyranoside (IPTG) to a final concentration of 0.1-3 mM, preferably 1 mM, which drove induction of GFP1-10 from pET GFP 1-10. Cells were returned to the incubating shaker for 0.5-2 hours, preferably 1 hour, to allow sufficient induction of GFP1-10. Cells were then sampled 1:10 into ice-cold phosphate buffered saline (Dulbecco's PBS, Teknova, Hollister, Calif.) containing 0.4 mg/mL chloramphenicol to halt protein synthesis. Cells were further diluted into the same saline solution to a density suitable for flow cytometry. Cells prepared in this manner were stable for up to 24 hours when stored at 4° C.

### Fluorescence-Activated Cell Sorting

Cells with maximal fluorescence were then selected by fluorescence-activated cell sorting (FACSaria II, BD, Franklin Lakes, N.J.). After proper setup of the cell sorter, following the manufacturer's instructions, sequentially induced cells were injected at a dilution appropriate for sorting, preferably 3,000 events/s. The subpopulation of sequentially-induced cells exhibiting the highest fluorescence, preferentially 1-5% of the total population, was then sorted. Sorting continued until the total number of cells injected exceeded effective library size determined supra.

### Cell Recovery

Recovered cells were concentrated by centrifugation across a Durapore PVDF 0.22 μm filter (Millipore, Ultrafree—MC #UFC30GVNB) at 1,000×g for 1 minute. The cells were washed with 500 μL cold PBS by centrifugation as before. 50 μL of recovery buffer (10 mM Tris pH 8.0, 0.1 mM EDTA) were added to the retained cells on the top of the filter and pipetted to resuspend the cells. The cells were freeze/thaw lysed by placing the filter in a −20° C. freezer for 30 minutes and then at room temperature for 30 minutes. The filter was then inverted into a clean 1.7 mL tube and centrifuged at 10,000×g for 1 minute to recover the buffer containing the lysed cells.

### Polynucleotide Recovery and Amplification

DNA encoding positively folding variants was recovered and amplified by PCR using tailed primers suitable for sub-cloning as follows: 0.3 μM forward and reverse primers, 10 μL buffer containing lysed cells, 25 μL 2×KOD Hot Start Master Mix (#71842-3, EMD Chemicals), water to 50 μL. Reactions were incubated at 95° C. for 2 minutes to activate the polymerase followed by 26 cycles of 95° C. for 20 seconds, 55° C. for 20 seconds, and 68° C. for 45 seconds. Resulting amplicons were purified by preparative agarose gel electrophoresis.

### Example 2

### Fluorescence-Activated Cell Sorting Control Proteins

Using the methods given in Example 1, positive (F+) and negative (NF) folding controls can be discriminated by fluorescence and used in an exemplary experiment for discrimination based on fluorescence intensity, which is a proxy for solubility. In this example, the positive folding control (F+) is E. coli Maltose Binding Protein (MBP), which is known in the art to be a highly soluble protein. The negative folding control (NF) in this example is cellobiohydrolase 2 from the filamentous fungus Hypocrea jecomia (née Trichoderma reesei), which has been demonstrated to express in insoluble form in E. coli. Both genes were cloned into the pTET GFP 11 vector system and separately transformed into E. coli cells harboring the pET GFP 1-10 vector and sequentially induced, as described in Example 1.

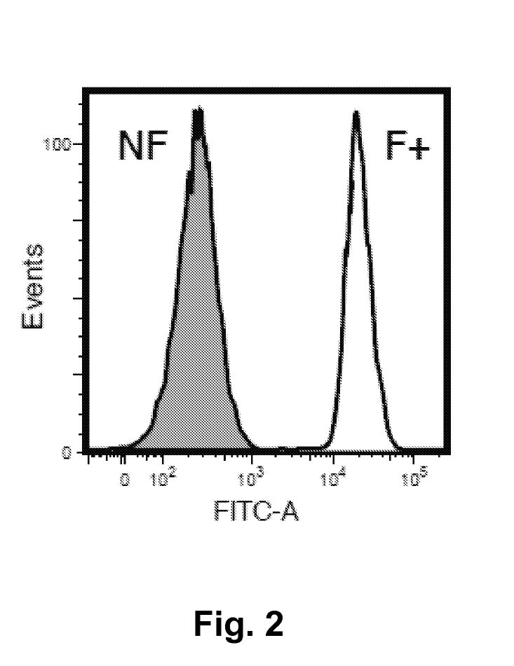

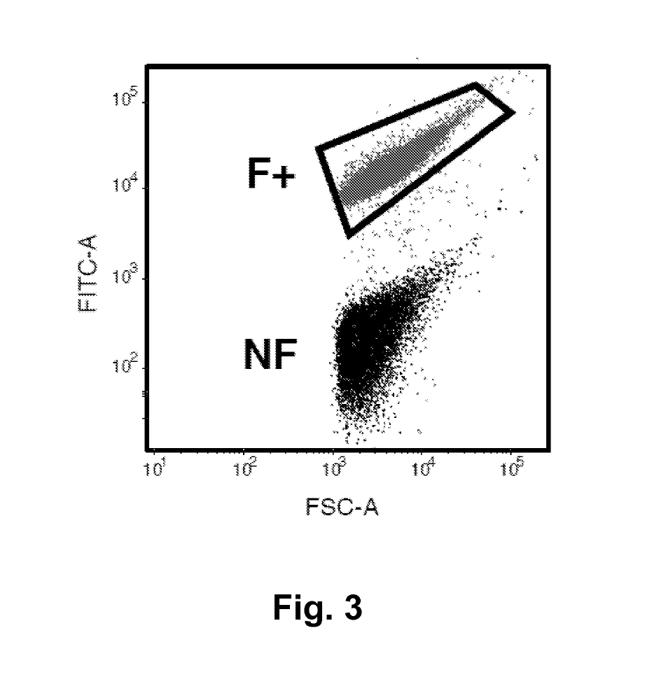

As presented in FIG. 2, populations of F+ and NF can be readily distinguished by their fluorescence, given as FITC-A. Further, the populations of F+ and NF can be readily sorted using fluorescence-activated cell sorting by those skilled in the art. FIG. 3 depicts an example gate that bounds the more fluorescent F+ population, as would be required in the operation of a FACS instrument.

### Example 3

### Fluorescence-Activated Cell Sorting Libraries

Using the methods given in Example 1, the activity of an error-prone library was enriched. In this example, an error-prone library (EPL) was generated as using the GeneMorph® II Random Mutagenesis kit #200550 (Agilent Technologies, Santa Clara, Calif., USA) follows. Thermoascus aurantiacus GH5 cDNA was used as template in 25 μL error-prone PCR reactions composed of 1×Mutazyme II buffer, 0.2 mM dNTP mix, 0.2 μM Primer C, 0.2 μM Primer D, 1.25 U Mutazyme II polymerase, and 4, 20, or 100 ng of template. The reaction was performed in an Mj Mini™ thermal cycler #PTC-1148 (Bio-Rad, Hercules, Calif., USA) programmed for 1 cycle at 95° C. for 2 minutes; and 30 cycles each at 95° C. for 30 seconds, 60° C. for 30 seconds, 72° C. for 1 minute. Then the reaction was incubated at 72° C. for 10 minutes. The error-prone reaction products were isolated by 0.8% low melting point agarose gel electrophoresis using TAE buffer (40 mM Tris, 20 mM acetic acid, and 1 mM EDTA) where approximately 1 kb product bands were excised from the gels and purified using a NucleoSpin® Extract II kit (Macherey-Nagel, Duren, Germany) according to the manufacturer's protocol. The purified products were cloned into the pTET GFP 11 vector system and transformed into E. coli cells harboring the pET GFP 1-10 vector and sequentially induced, as described in Example 1. Separately, wild-type (WT) and a known insoluble Thermoascus aurantiacus GH5 variant (IV) was cloned into the pTET GFP 11 vector system and transformed into E. coli cells harboring the pET GFP 1-10 vector and sequentially induced, as described in Example 1.

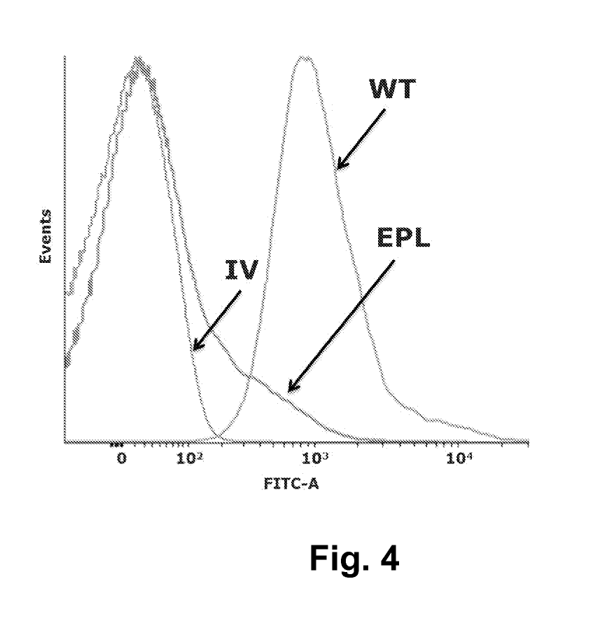

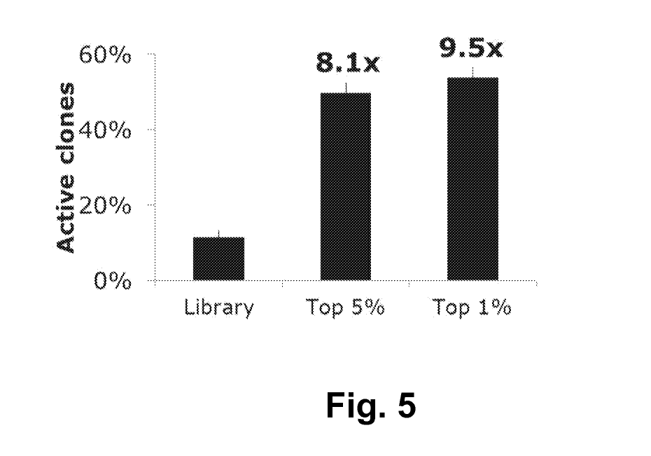

As presented in FIG. 4, the error-prone library (EPL), wild-type (WT), and insoluble variant (IV) can be distinguished by their fluorescence profiles, given as FITC-A. Further, sorting either the highest 5% or 1% fluorescing cells using fluorescence-activated cell sorting by those skilled in the art, results in 8.1-fold and 9.5-fold increases in the number of active clones in the library (FIG. 5).

1. A method of enriching a plurality of polynucleotides for polynucleotides likely to encode functional polypeptides and screening the enriched plurality, the method comprising:
(a) providing a plurality of polynucleotides encoding variants of a polypeptide, wherein at least some of the polynucleotides encode a polypeptide having at least one activity;
(b) producing polypeptides from the polynucleotides;
(c) determining whether the polypeptides are soluble;
(d) selecting the polynucleotides that encode soluble polypeptides to form an enriched plurality of polynucleotides; and
(e) screening the enriched plurality for polynucleotides encoding polypeptides having the activity.

2. The method of claim 1, wherein said selecting produces an at least 2-fold enrichment in polynucleotides that encode a polypeptide having the activity or wherein said selecting produces a degree of enrichment selected from the group consisting of at least: 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold , 10³-fold, 10⁴-fold, 10⁵-fold, 10⁶-fold, 10⁸-fold, and 10⁹-fold enrichment in polynucleotides that encode a polypeptide having the activity.

3. A method of comparing at least two libraries of polynucleotides with respect to the level of polynucleotides likely to encode functional polypeptides, the method comprising:
(a) providing at least two different libraries of polynucleotides encoding variants of the same polypeptide;
(b) for each plurality:
(i) producing polypeptides from the polynucleotides; and
(ii) determining whether the polypeptides are soluble; and
(c) identifying the plurality that has the highest level of soluble polypeptides as the one that contains the highest level of polynucleotides likely to encode functional polypeptides.

4. The method of claim 3, wherein each plurality comprises at least some polynucleotides encoding a polypeptide having at least one activity, and the method additionally comprises screening the identified plurality for polynucleotides encoding polypeptides having the activity.

5. The method of claim 1, wherein the producing of (b) comprises expressing the polypeptides in a host cell.

6. The method of claim 5, wherein the polypeptides are expressed as fusion proteins in a host cell, wherein the fusion protein also comprises a solubility reporter portion.

7. The method of claim 6, wherein the host cell expresses a complementation polypeptide that is capable of binding to the solubility reporter portion of fusion proteins to produce a detectable protein complex.

8. The method of claim 1, wherein the producing of (b) comprises expressing the polypeptides in a reaction mixture comprising components for in vitro transcription/translation.

9. The method of claim 8, wherein the polypeptides are expressed as fusion proteins, wherein each fusion protein comprises solubility reporter portion(s) comprising:
a polypeptide attachment tag that is capable of forming a covalent bond with, or otherwise binding to, a polynucleotide of the plurality; and
a polypeptide affinity tag.

10. A method of screening for chaperones that facilitate protein folding, the method comprising:
(a) expressing a fusion protein in a host cell or in an in vitro reaction mixture, wherein the fusion protein comprises a portion that tends to misfold or aggregate, wherein the portion is linked to one or more solubility reporter portion(s), and wherein the host cell or in vitro reaction mixture also comprises or produces a potential chaperone;
(b) determining whether the fusion protein is soluble;
(c) if the fusion protein is soluble, identifying the potential chaperone as one that facilitates folding of the fusion protein.

11. The method of claim 10, wherein the fusion protein is expressed in a plurality of host cells or in vitro reaction mixtures, and different host cells or reaction mixtures, respectively, comprise or produce different potential chaperones, and wherein, when the fusion protein is expressed in in vitro reaction mixtures, at least about 20% of the reaction mixtures comprise or produce one or fewer of said potential chaperones per reaction mixture.

12. The method of claim 11, wherein the potential chaperones are expressed from a polynucleotide plurality selected from a plurality encoding known chaperone polypeptides, a plurality encoding variants of one or more known chaperone polypeptides, a plurality encoding peptides, a plurality derived from a sample from a plant or animal or an environmental sample, or the potential chaperones a comprise small-molecule plurality selected from a plurality of known small-molecule chaperones, a plurality of variants of one or more known small-molecule chaperones, and a plurality of small molecules, wherein each small molecule in the small-molecule plurality is linked to a unique polynucleotide barcode.

13. The method of claims 11, wherein the fusion protein is expressed in host cells that express a complementation polypeptide that is capable of binding to the solubility reporter portion of fusion proteins to produce a detectable protein complex.

14. The method of claims 11, wherein the fusion proteins are expressed in separate reaction mixtures that comprise aqueous phase droplets in a water-in-oil emulsion.

15. The method of claim 14, wherein each fusion protein comprises solubility reporter portion(s) comprising:
a polypeptide attachment tag that is capable of forming a covalent bond with, or otherwise binding to, a polynucleotide; and
a polypeptide affinity tag.

16. A method of protein domain mapping to identify one or more soluble and/or functional domain(s), the method comprising:
(a) expressing a fusion protein in a host cell or in an in vivo reaction mixture, wherein the fusion protein comprises a portion of a protein to be mapped, wherein the portion is linked to one or more solubility reporter portion(s);
(b) determining whether the fusion protein is soluble;
(c) if the fusion protein is soluble, identifying the portion as one that is a soluble and/or functional domain.

17. The method of claim 16, wherein a plurality of different fusion proteins is expressed in a plurality of host cells or in vivo reaction mixtures, and wherein, when the fusion protein is expressed in in vitro reaction mixtures, at least about 20% of the reaction mixtures comprise or produce one or fewer of said potential chaperones per reaction mixture.

18. The method of claim 17, wherein the fusion proteins are expressed in a host cell that expresses a complementation polypeptide that is capable of binding to the solubility reporter portion of soluble fusion proteins to produce a detectable protein complex.

19. The method of claim 17, wherein the fusion proteins are expressed from polynucleotides encoding the fusion proteins in separate reaction mixtures that comprise aqueous phase droplets in a water-in-oil emulsion.

20. The method of claim 19, wherein each fusion protein comprises solubility reporter portion(s) comprising:
a polypeptide attachment tag that is capable of forming a covalent bond with, or otherwise binding to, a polynucleotide encoding the fusion protein; and
a polypeptide affinity tag.
